SEQUENCE LISTING (4 sequences)

SEQ ID NO: 1 - DNA, 963 bp, Artificial Sequence
  Nucleotide sequence of SUMF2 gene
atgagggcgcatgcgcagcggggccgtgggtgtacgcggcgcagcgcggcagtcctgatggcccggcatgggttaccgctgctgcccctgctgtcgctcctggtcggcgcgtggctcaagctaggaaatggacaggctactagcatggtccaactgcagggtgggagattcctgatgggaacaaattctccagacagcagagatggtgacgggcctgtgcgggaggcgacagtgaaaccctttgccatcgacatatttcctgtcaccaacaaagatttcagggattttgtcagggagaaaaagtatcggacagaagctgagatgtttggatggagctttgtctttgaggactttgtctctgatgagctgagaaacaaagccacccagccaatgaagtctgtactctggtggcttccagtggaaaaggcattttggaggcagcctgcaggtcctggctctggcatccgagagagactggagcacccagtgttacacgtgagctggaatgacgcccgtgcctactgtgcttggcggggaaaacgactgcccacggaggaagagtgggagtttgccgcccgagggggcttgaagggtcaagtttacccatgggggaactggttccagccaaaccgcaccaacctgtggcagggaaagttccccaagggagacaaagctgaggatggcttccatggagtctccccagtgaatgctttccccgcccagaacaactacgggctctatgacctcctggggaacgtgtgggagtggacagcatcaccgtaccaggctgctgagcaggacatgcgcgtcctccggggggcatcctggatcgacacagctgatggctctgccaatcaccgggcccgggtcaccaccaggatgggcaacactccagattcagcctcagacaacctcggtttccgctgtgctgcagacgcaggccggccgccaggggagctgtaa

SEQ ID NO: 2 - DNA, 927 bp, Artificial Sequence
  Sequence of mouse SUMF2 cDNA
atgcgctctgagttctggttccccagcatgggttccttgctccctccggtgttgctgctgtggctcctgtcctgccccaggcttcagctaggacatgcccaggatcctgccatggtgcatctgccaggtggccggtttctgatggggacagacgctccagatggcagagacggtgaagggcctgcccgggaagtgacagtaaaaccctttgccatcgacatatttccagtcaccaataaagacttcagggagtttgtcagggagaagaagtaccagactgaagccgaggcattcgggtggagcttcgtctttgaggattttgtctcccctgagctcagaaagcaagaaaatctgatgccggctgttcactggtggcagccagtgccaaaggcattttggaggcagcctgcaggtcccggctctggcatccgagagaaactggagcttcccgtggtacacgtgagctggaacgacgctggtgcttactgcgcatggcgggggagacgcttgcccacagaagaggagtgggagtttgcagcccgagggggcttgaagggtcaggtttatccatgggggaaccggttccagccaaaccgcaccaacttatggcagggaaagttccccaaaggtgacaaagctgaagatggttttcatggactgtcaccagtgaacgctttccccccacagaacaactacggactgtatgacctcatgggcaatgtgtgggagtggacagcgtccacataccaacctgctggccaggacatgcgtgtcctccggggggcatcatggatcgacaccgcagacggctctgctaatcacagggctcgggtcaccaccaggatgggaaacactccagactcagcctcagacaacctgggcttccgctgcgcctccagtgcaggccgaccgaaggaggacctgtga

SEQ ID NO: 3 - DNA, 20 bp, Artificial Sequence
  Forward primer for PCR reaction
acgctatgtggatacgctgc

SEQ ID NO: 4 - DNA, 20 bp, Artificial Sequence
  Reverse primer for PCR reaction
cgggccacaactcctcataa

(19) United States
(12) Patent Application Publication
(10) Pub. No.: US 2022/0401582 A1
(43) Pub. Date: Dec. 22, 2022

(54) USE OF SUMF2 GENE AS A GENE THERAPY TARGET FOR PREVENTING AND/OR TREATING ALLERGIC ASTHMA ATTACK AND REDUCING AIRWAY HYPERRESPONSIVENESS

(71) Applicant: Harbin Institute of Technology, Harbin (CN)

(72) Inventors: Meng JIANG, Harbin (CN); Xiaofeng JIANG, Harbin (CN); Huijun GAO, Harbin (CN); Bin LI, Harbin (CN); Wenxuan SUN, Harbin (CN); Ye ZHENG, Harbin (CN); Mingsi TONG, Harbin (CN)

(21) Appl. No.: 17/571,888

(22) Filed: Jan. 10, 2022

(30) Foreign Application Priority Data
Jun. 11, 2021 (CN) 202110652057.X

(51) Int. Cl. A61K 48/00; A61K 38/17; A61P 11/06; A61P 37/08

(52) U.S. Cl. CPC A61K 48/005; A61K 38/1709; A61K 48/0008; A61P 11/06; A61P 37/08

(57) ABSTRACT

Provided is the use of as SUMF2 gene therapy target for preventing and/or treating an allergic asthma attack and reducing airway hyperresponsiveness. It is verified that the abnormal metabolic pathway of SUMF1/GAG causes epithelial cells to produce a similar immune memory and the epithelial cells can promote T_{H}2 response when exposed to allergens again. In the present disclosure, SUMF2 gene in airway epithelial cells from a mouse is overexpressed using the adeno-associated virus vector 20 days before the induction of asthma. Compared with a control group, the present disclosure can significantly improve the lung airway inflammation in an asthmatic mouse. Therefore, the overexpression of SUMF2 can be used as a gene therapy target to prevent allergic asthma and reduce airway hyperresponsiveness. Therefore, the use of an adeno-associated virus to overexpress SUMF2 for the prevention of allergic asthma has broad use values and market prospects.

### CROSS REFERENCE TO RELATED APPLICATION

This patent application claims the benefit and priority of Chinese Patent Application No. 202110652057.X filed with China National Intellectual Property Administration on Jun. 11, 2021, the disclosure of which is incorporated by reference herein in its entirety as part of the present application.

### REFERENCE TO SEQUENCE LISTING SUBMITTED ELECTRONICALLY

A sequence listing electronically submitted with the present application as an ASCII text file named GWP202107341SequenceListing.txt, created on 11-5-2021 and having a size of 4000 bytes, is incorporated herein by reference in its entirety.

### TECHNICAL FIELD

The present disclosure belongs to the field of biomedical technology, and specifically relates to the use of a Sulfatase Modifying Factor 2 (SUMF2) gene as a gene therapy target for preventing and/or treating an allergic asthma attack and reducing airway hyperresponsiveness.

### BACKGROUND

Allergic asthma is a special type of bronchial inflammation characterized by T_{H}2 response. At present, the main treatment approaches of asthma are the use of bronchodilators, leukotriene receptor antagonists and steroid drugs. Severe grade-3 or higher asthma requires the addition of long-acting cholinergic receptor antagonists such as anti-IgE, anti-IL-5 or anti-IL-4R. These drugs are mainly used to reduce airway inflammation and relieve smooth muscle spasm following the onset of asthma but have limited effect on inflammatory injury induced airway remodeling. Although the treatment can delay the progression of asthma, the airway remodeling caused by inflammation is irreversible and will eventually cause irreversible airflow restriction. Therefore, in addition to conventional treatment methods such as inflammation suppression and smooth muscle relaxation, there is an urgent need for a clinical treatment approach to reduce the number of attacks for patients and further fundamentally inhibit the inflammation induced airway remodeling.

### SUMMARY

In the view of this, the purpose of the present disclosure is to provide the use of SUMF2 gene as a gene therapy target for preventing and/or treating an allergic asthma attack and reducing airway hyperresponsiveness. Highly expressed SUMF2 gene increases the minimum threshold of T_{H}2 inflammation initiation in airway epithelial cells (AECs), thereby preventing asthma attack and reducing airway hyperresponsiveness.

The present disclosure provides use of SUMF1/2 as a gene therapy target for preventing and/or treating an allergic asthma attack and reducing airway hyperresponsiveness.

In one embodiment of the present disclosure, SUMF2 gene has a nucleotide sequence as shown in SEQ ID NO: 1.

In one embodiment of the present disclosure, SUMF2 gene is used jointly with a gene therapy vector for preventing and/or treating allergic asthma attack and reducing airway hyperresponsiveness.

In one embodiment of the present disclosure, the gene therapy vector includes an adeno-associated virus packaging system.

In a further embodiment of the present disclosure, the adeno-associated virus packaging system includes a shuttle plasmid for inserting exogenous genes, a pAAV6-RC plasmid for encoding Rep and Cap proteins, and a pHelper plasmid for expressing adenovirus proteins.

The present disclosure provides a gene therapy target for preventing and/or treating an allergic asthma attack and reducing airway hyperresponsiveness, including SUMF2 gene in airway epithelial cells.

In one embodiment of the present disclosure, the gene therapy target further includes a gene therapy vector for SUMF2 gene.

In one embodiment of the present disclosure, the gene therapy vector includes an adeno-associated virus packaging system.

In one embodiment of the present disclosure, the adeno-associated virus packaging system includes a shuttle plasmid for inserting exogenous genes, a pAAV6-RC plasmid for encoding Rep and Cap proteins, and a pHelper plasmid for expressing adenovirus proteins.

The present disclosure provides the use of SUMF2 gene as a gene therapy target for preventing and/or treating an allergic asthma attack and reducing airway hyperresponsiveness. Current data revealed that the glycosaminoglycan (GAG) metabolism in airway epithelial cells (AECs) changed as the increase of the relative expression level of SUMF1/2 when asthma occurs. This change cannot be reversed to normal even if the allergen is removed and the T_{H}2 reaction subsides for a few weeks. When the body is exposed to the allergen again, the continuously activated presentation pathway for a lysosome-major histocompatibility complex (MHC) class II antigen as well as a cytokine free from restriction by GAG will exacerbate the AEC-mediated inflammatory response. In view of this, in the present disclosure, the expression of lysosome and MHC II-related genes and the metabolism of GAG in the lysosome are inhibited by targeting and overexpressing the SUMF2 gene in airway epithelial cells, thereby blocking the T_{H}2 inflammatory responses. The overexpression of SUMF2 gene elevates the threshold of allergic reaction initiation in AECs, thereby preventing asthma attacks and reducing airway hyperresponsiveness.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1C show the bioinformatics analysis of airway epithelial cells from allergic rhinitis/asthma patients, where FIG. 1A shows the bioinformatics analysis process; FIG. 1B and FIG. 1C show correlation analysis of a gene set; Heatmap represents the Pearson correlation coefficient between the characteristic values for 14 conservative modules E1-E14 in the Consensus work in different sample sets and in the signal pathway gene set shown in the figure. Red represents positive correlation, blue represents negative correlation; H-S represents Heparan sulfate; K-S represents Keratan sulfate, C-S represents Chondroitin sulfate; D-S represents Dermatan sulfate; and H represents Hyaluronic acid.

FIG. 2A-FIG. 2D show the result for supervised cluster analysis of airway epithelial cells in asthma patients, where scatter plots show the characteristic values of the E1-E14 gene sets in a patient with allergic rhinitis, and gene pathways that have the greatest Pearson correlation with the characteristic value of the current gene set are shown under the scatter plots.

FIG. 3A-FIG. 3F show a pseudo-time series of airway allergic diseases by bioinformatics analysis, where FIG. 3A represents a Consensus network consisting of conservative gene regulatory relationships in 5 sets of expression profile data; each node represents a gene set whose regulatory relationship is relatively conservative among the 5 sets of expression profile data; connection lines between the nodes represent different Pearson correlation between the characteristic values of the gene set, red lines represent a positive correlation, and blue lines represent a negative correlation. Width of the lines represents absolute value of the correlation, and thicker lines represent gene sets with higher correlation; FIG. 3B is a pseudo time series of Potential of Heat-diffusion for Affinity-based Trajectory Embedding (PHATE) showing the evolution of the epithelial cell expression profile of a patient with allergic rhinopathy. Each spot in the scatter plot represents a patient, color of the scatter plot represents a group of patients with similar expression profiles, and arrows indicate the change direction of the two most significant characteristics in the expression profile. FIG. 3C is a schematic diagram of the regulation of sulfatase activity by SUMF1/2 (upper scheme) and a schematic diagram of heparan sulfate metabolism (lower scheme). FIG. 3D shows the scatter plot of the expression level of the shown gene changing with the pseudo time series. FIG. 3E shows the ratio of Reads in RNA-seq of a mouse SUMF1/2 under different conditions. FIG. 3F shows the RNA-seq result of Sgsh and Naglu under different conditions, and P value is calculated by Student's t-test: * denotes P<0.05, ** denotes P<0.01, and n.s. represents that the difference is not significant.

FIG. 4A-FIG. 4E show the expression level of SUMF1/2 and airway inflammation. FIG. 4A shows the modeling method for ovalbumin (OVA)-induced asthma in mice; FIG. 4B shows the modeling method for lipopolysaccharide (LPS)-induced acute lung injury in mice; FIG. 4C shows the result for hematoxylin-eosin (H&E), Periodic Acid-Schiff (PAS) and immunohistochemistry (IHC) staining of mouse lungs under different conditions, with the scale bars being as follows: 250 μm for H&E staining, and 50 μm for PAS staining and SUMF1/2 IHC staining, and 10 μm for H-S Epitope 10E4; FIG. 4D is a construction method of LPS-pretreated asthma model in a mouse. FIG. 4E shows the results for H&E, PAS and IHC staining of mouse lungs under different conditions, with the scale bars being as follows: 250 μm for H&E staining, and 50 μm for PAS staining and IHC staining.

FIG. 5AA-FIG. 5F show a gene therapy targeting SUMF1/2 to increase the airway allergic reaction threshold FIG. 5A is a schematic diagram of AAV6-mSUMF2 infecting airway epithelial cells of a mouse by nebulized inhalation. FIG. 5B is a schematic diagram of the construction method of a mouse asthma model and the administration time for AAV treatment (upper panel), and the Western blotting shows the expression level of recombinant mouse SUMF2-Flag in airway epithelium (lower panel). FIG. 5C is the IHC staining, showing the ability of AAV5, AAV6 and AAV9 to infect different types of lung cells, where red arrows indicate SUMF2 positive cells and the scale bar is 100 μm. FIG. 5D shows the inhibitory effects of different AAV6-mSUMF2 on airway allergic reactions in different states, detected by H&E, IHC and computed tomography (CT) scans, where black triangles in IHC staining indicate the staining of HS 10E4 in the basement membrane direction of airway epithelial cells; the scale bar is 250 μm for H&E staining, and 10 μm for IHC staining. FIG. 5E is a histogram reflecting the percentage of AAV6-mSUMF2 on airway cross-sections showing different infiltration levels of immune cells in lung tissues of mice under different conditions; P value is calculated by chi-square test: and the symbol **** denotes P<0.0001, and n.s. represents non-significant difference is. FIG. 5F shows the immunofluorescence staining of lung tissues of the mice treated with AAV6-mSUMF2-treated and of mice in the control group after OVA stimulation; red represents GATA3, green represents T-bet, and blue represents cell nucleus; white triangles indicate GATA3+ cells, and the scale bars are: 100 μm (left), and 20 μm (right).

FIG. 6A-FIG. 6D show the inhibition of lysosome function by AAV6-mSUMF2 gene therapy-MHC II antigen presentation, where FIG. 6A, FIG. 6B and FIG. 6C show the functional enrichment analysis (left) and the differential gene expression profile Heatmap (right) of the differentially-expressed genes in lung tissue of mice in the AAV6-mSUMF2 gene-treated mouse and in the control group under different conditions. FIG. 6D shows the immunofluorescence staining profile of the airway epitheliums of the mice treated with AAV6-mSUMF2 gene and of the mice in the control group following OVA stimulation, wherein green represents MHC II, red represents LAMP1, blue represents cell nucleus, white arrows indicate co-localization signal of MHC II and LAMP1, and the scale bar is 5 μm for white, and 2 μm red.

FIG. 7 is the diagram of a recombinant shuttle plasmid constructed by the method of present disclosure, in which is SUMF2-3flag/3flag gene fragment is inserted.

FIG. 8 is the diagram of a pHBAAV-Cap6 plasmid in the adeno-associated virus packaging system in the examples of the present disclosure.

FIG. 9 is the standard curve for determination of AAV6 virus titer in the examples of the present disclosure.

### DETAILED DESCRIPTION OF THE EMBODIMENTS

The present disclosure provides the use of SUMF2 gene as a gene therapy target for preventing and/or treating an allergic asthma attack and reducing airway hyperresponsiveness.

In the present disclosure, SUMF2 gene preferably has a nucleotide sequence shown in SEQ ID NO: 1.

In the present disclosure, SUMF2 treatment nearly blocks all events related to the T_{H}2 response by overexpressing SUMF2 in the lung airway epithelial cells in the allergic asthma mouse model. Meanwhile, SUMF2 treatment can inhibit the expression of lysosomes and MHC II pathway associated genes, while reducing the punctate staining of MHC II and leading lysosomal accumulation. The sulfatase in lysosomes is inhibited by AAV6-mSUMF2, which may be the reason of lysosome accumulation. Therefore, the present disclosure further provides the use of SUMF2 gene as the gene therapy target for preventing and/or treating the allergic asthma attack and reducing airway hyperresponsiveness.

In the present disclosure, SUMF2 gene is preferably used jointly with a gene vector to prevent and/or treat the allergic asthma attack and reducing airway hyperresponsiveness. Preferably, the gene vector includes an adeno-associated virus packaging system. Preferably, the adeno-associated virus packaging system includes a shuttle plasmid for inserting exogenous genes, a pAAV6-RC plasmid for encoding proteins Rep and Cap, and a pHelper plasmid for expressing adenovirus proteins. In the examples of the present disclosure, the pAAV6-RC plasmid (pHBAAV-Cap6) encoding proteins Rep and Cap and the pHelper plasmid expressing adenovirus proteins are purchased from Hanbio Company.

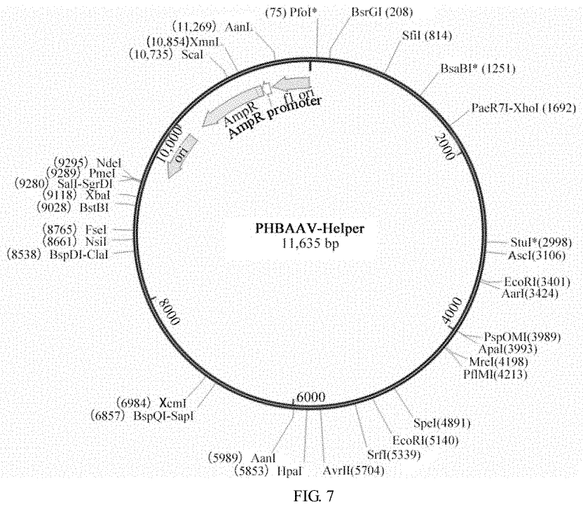

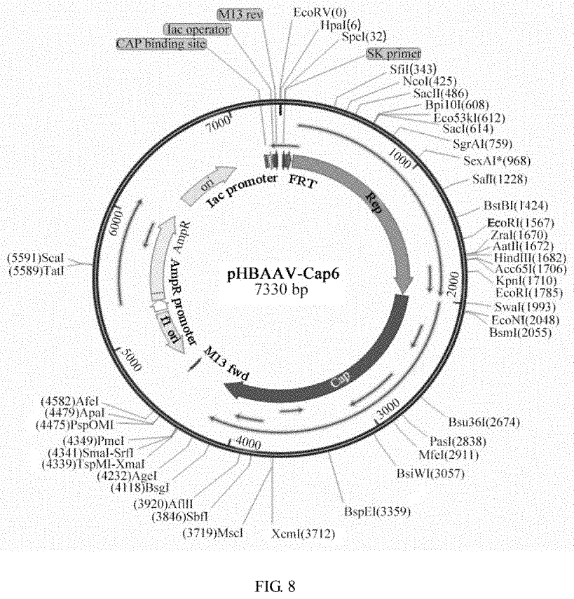

In the present disclosure, when SUMF2 gene is used jointly with a gene vector to prepare the therapeutic adeno-associated virus, it is preferred that SUMF2 gene is inserted into a shuttle plasmid to form a recombinant shuttle plasmid. In the present disclosure, there is no specific limitation to the method for constructing the recombinant shuttle plasmid, and the construction method well known in the art may be employed. FIG. 7 shows a recombinant shuttle plasmid constructed as such. In the present disclosure, an AAV6 subtype that can advantageously target mouse lung airway epithelial cells to overexpress SUMF2 is selected. The AAV6 genome includes two inverted terminal repeats (ITR) and two open reading frames (ORF), namely Cap and Rep, and Rep can be translated into a variety of proteins necessary for the life cycle of AAV. In the AAV packaging system, Rep and Cap are substituted with the target gene SUMF2-3flag/3flag, and then co-transfects a 293T cell along with a plasmid expressing the Rep and Cap genes to form an AAV virus particle which carries a foreign inserted gene. FIG. 8 shows a diagram of the AAV packaging system. Preferably, the method of co-transfection includes the following steps: mixing a 293T cell having a confluence of 50-70% and being free of mycoplasma contamination with a viral plasmid for virus packaging, and transfecting a system in a T75 culture flask containing the following components: AAV-RC 10 μg, Helper 20 μg, AAV-SUMF2 10 μg, and LipoFiter 80 μl. After 6 hours of transfection, continue the culturing by transferring the system to a DMEM medium containing 10% fetal bovine serum, collecting all the cells after 72 hours, freezing and thawing the cells three times using liquid nitrogen and collecting the supernatant; purifying the supernatant with a Biomiga purification column, and digesting the target sample of AAV virus with DNase I and proteinase K, determining the titer of the virus by real-time fluorescent quantitative PCR (using a plasmid having a known copy number as a standard), dispensing and storing the sample at −80° C.

In the present disclosure, the AAV6-SUMF2 has the property of targeting lung airway epithelial cells. Introducing SUMF2 gene into the animal body along with the adeno-associated virus enables SUMF2 to overexpress in airway epithelial cells, which significantly decreases the infiltration of immune cells as well as reversing the GAG sulfation on the cell surface, thereby inhibiting the T_{H}2 allergic reaction.

The present disclosure provides a gene therapy for preventing and/or treating an allergic asthma attack and reducing airway hyperresponsiveness, including SUMF2 gene in airway epithelial cells. Preferably, the gene therapy further includes a gene vector of SUMF2 gene. Preferably, the gene vector includes an adeno-associated virus packaging system. Preferably, the adeno-associated virus packaging system includes a shuttle plasmid for inserting exogenous genes, a pAAV6-RC plasmid for encoding Rep and Cap proteins, and a pHelper plasmid for expressing adenovirus proteins. In the present disclosure, there is no particular limitation to a dosage form of the drug for the gene therapy, and dosage forms of the drug well known in the art may be adopted. Experiments in the present disclosure prove that specific overexpression of SUMF2 gene in lung airway epithelial cells can effectively increase the threshold of AECs to trigger an allergic reaction, thereby preventing asthma attack and reducing airway hyperresponsiveness.

The use of SUMF2 gene therapy for preventing and/or treating an allergic asthma attack and reducing airway hyperresponsiveness provided by the present disclosure will be described in detail below with reference to examples, but it should not be understood as limiting the protection scope of the present disclosure.

### Example 1

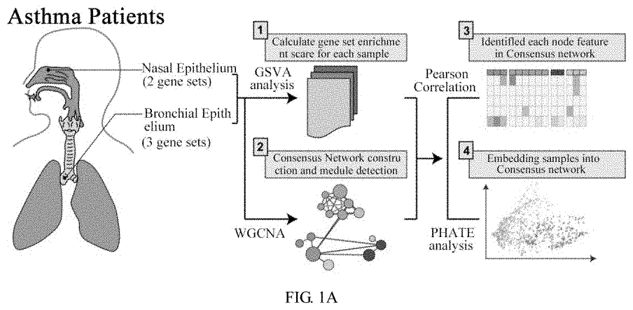

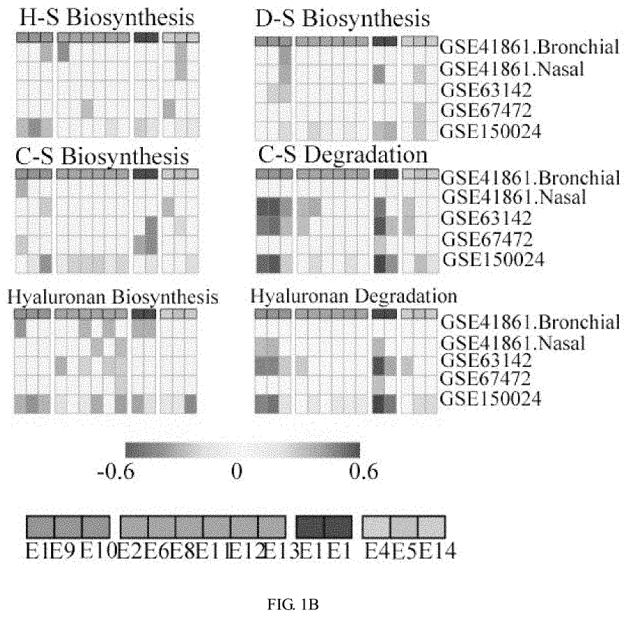

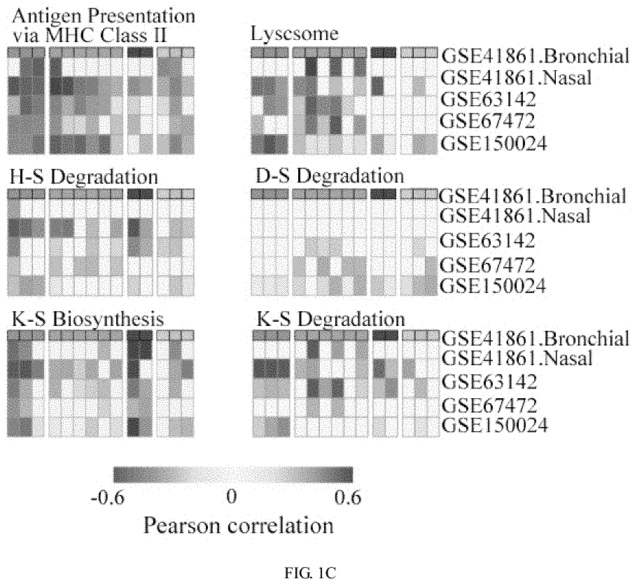

In this example, a screening method was provided for identifying SUMF1/2 as a therapy target in allergic asthma patient. An asthma time series model was established using bioinformatics analysis, and the analysis process is shown in FIG. 1a.

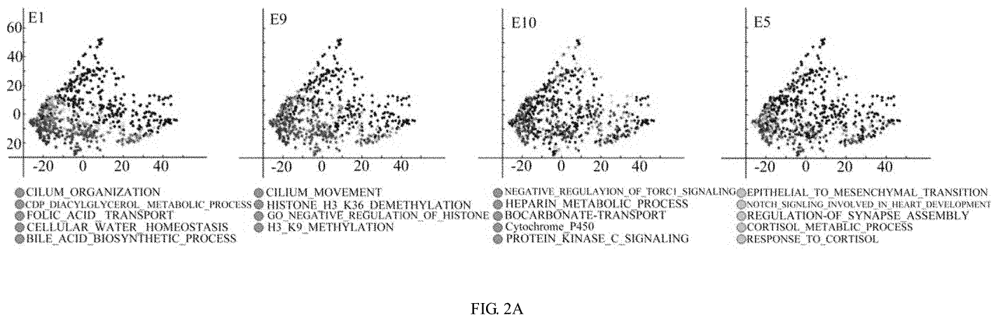

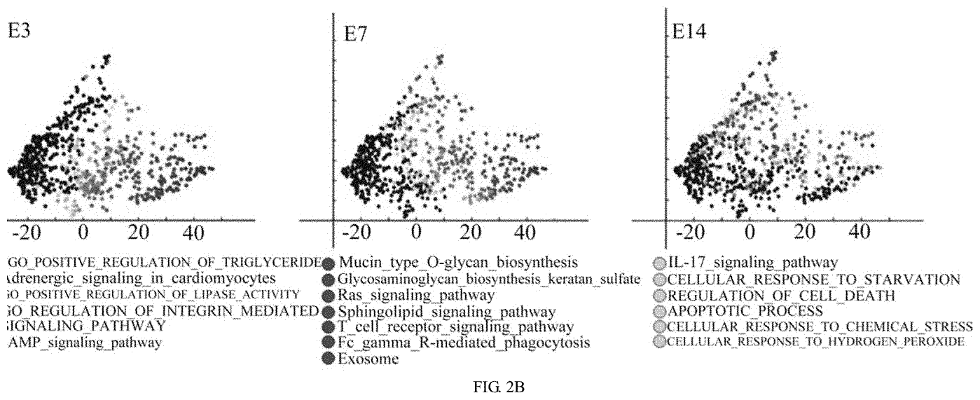

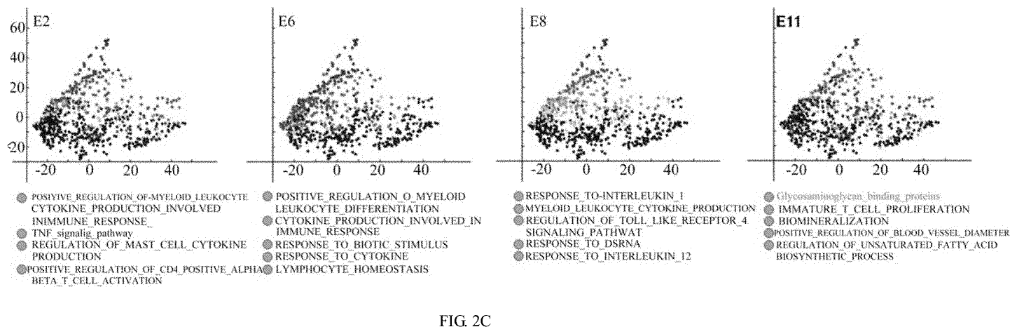

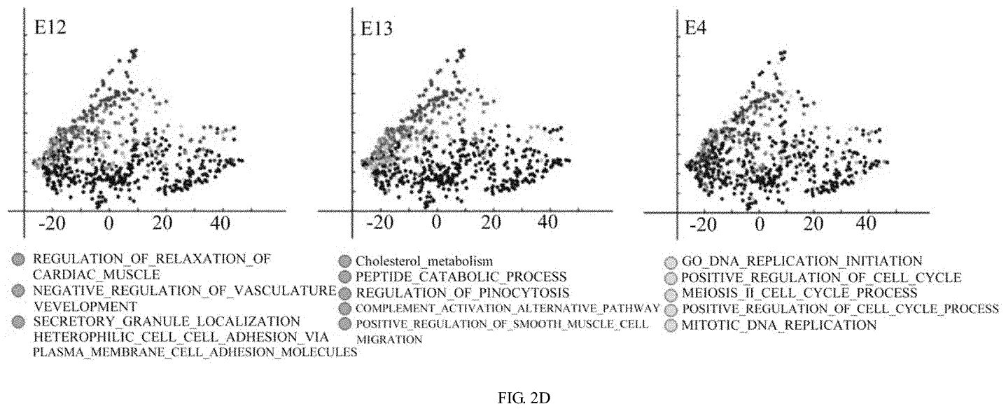

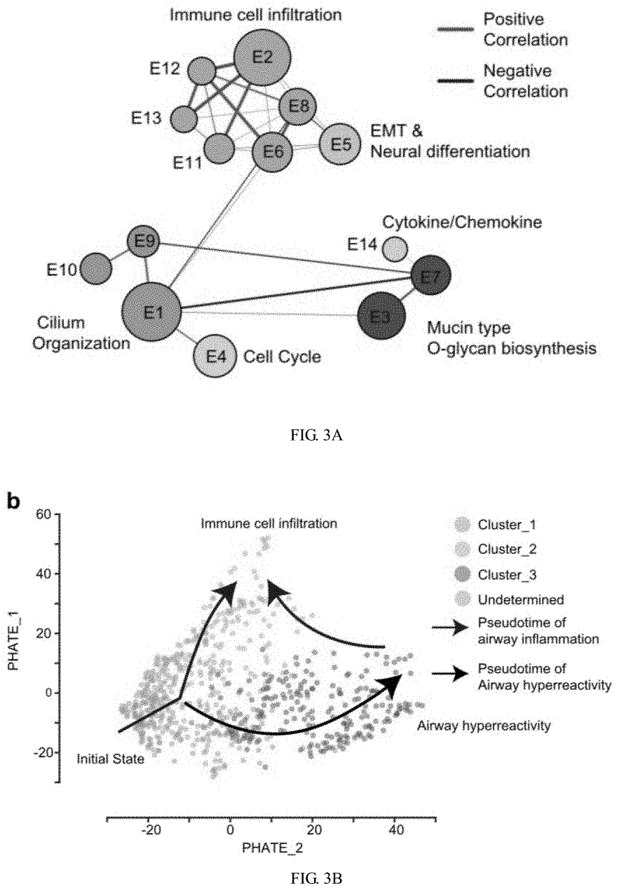

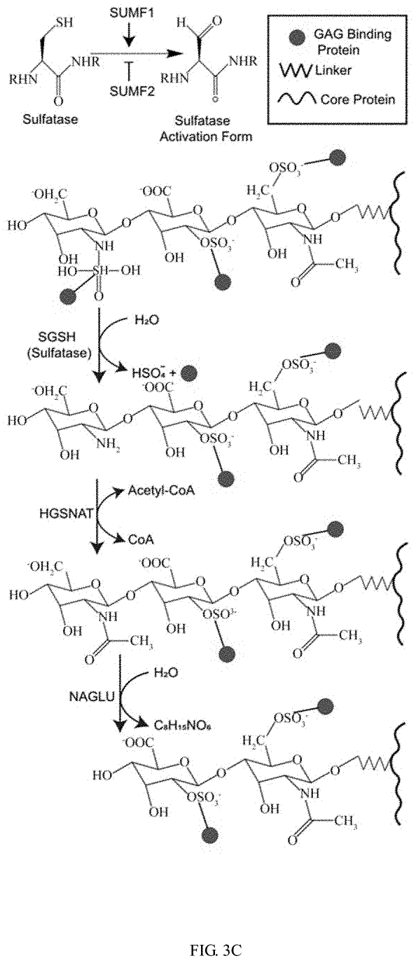

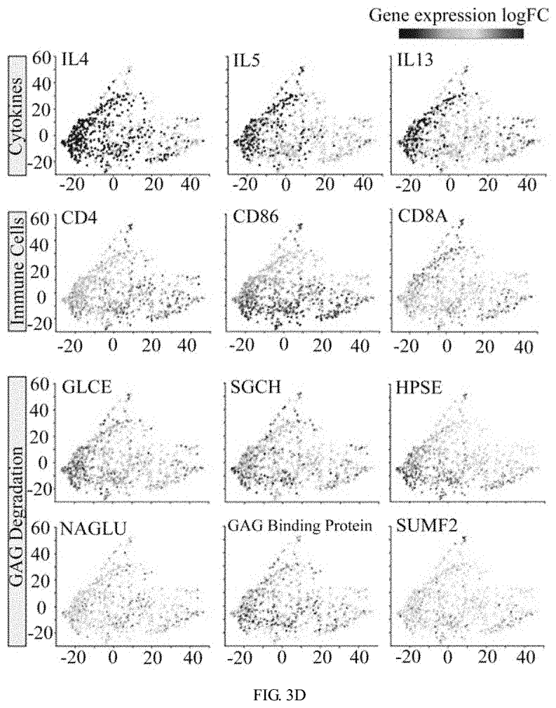

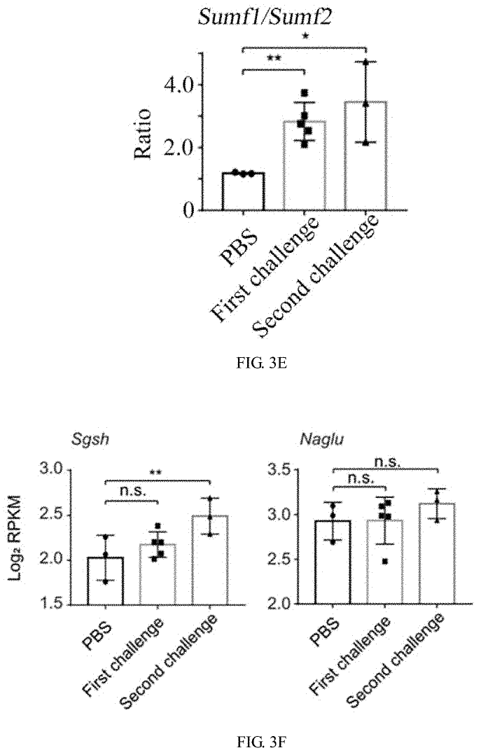

The RNA-seq and microarray data on nasal mucosa and airway epithelium of the asthmatic patients were subjected to fuzzification processing. Fisrtly, the dimensionality of the raw data was reduced using a Gene Set Variation Analysis (GSVA) method to transform the data on the expression profile from expression values of a single gene to an enrichment degree of different signal pathways, giving a cell pathway enrichment matrix. Secondly, weighted gene co-expression network analysis (WCGNA) was conducted on 5 sets of RNA-seq and microarray data from different samples, and diagrams of gene expression regulation network were generated using the genes as nodes and Pearson correlation of expression values between the genes as weights. A Consensus Network diagram was generated by selecting the common parts of the five sets of gene expression regulation network diagrams, in which Consensus Network represented a conservative inter-gene regulation relationship that existed both in different data sets and in different diseased sites in the entire airway mucosa. In the Consensus Network diagram, each node represented a set of gene modules having a mutually regulated relationship, and the connection lines between the nodes represented the correlation between the nodes. When the genes constituting the nodes were used as a characteristic vector, a characteristic value of the characteristic vector could be obtained by a least square method among the different data sets and the different samples, and a correlation analysis was conducted against the characteristic values and the GSVA results to give a biological event represented by the node. Once again, manifold dimensionality reduction was conducted on the gene sets represented by the nodes, using a PHATE algorithm. Due to the nature that PHATE reorders the samples, ordering of samples after dimensionality reduction by the algorithm was a pseudo-time series that can represent the biological process. According to the distribution of different gene sets in the samples, which is shown in FIG. 2, molecular biological changes in the process of asthma could be plotted in FIG. 3b.

The results are shown in FIG. 1b and FIG. 1c, a total of 14 conservative modules E1-E14 were generated through WCGNA analysis, representing a gene set having a very stable mutual regulation relationship in allergic asthma. Further analysis of the gene sets related to these nodes revealed that all types of GAG degradation pathways, except for hyaluronic acid, were positively correlated with asthma. Likewise, the positively correlated signal pathways further include MHC II antigen presentation and lysosomal signaling.

Pearson correlation analysis was conducted on 14 conservative gene modules, and the signal pathways having the strongest Pearson correlation to 1-14 modules were selected from the cell pathway enrichment matrix following GSVA dimensionality reduction as functional labels for the conservative modules. As shown in FIG. 2, the network containing 14 modules was correlated to three biological events: (1) ciliated epithelium-related genes; (2) immune cell infiltration; (3) mucopolysaccharide synthesis and metabolism. These three biological events exactly correspond to the three landmark events in allergic asthma.

The results for the manifold analysis are shown in FIG. 3a and FIG. 3b, and each point in the scatter plots represents a patient sample. The clustering analysis of Consensus cluster was conducted on a sample expression profile in the scatter plot to obtain three main subtypes, representing the three most important characteristics in the attack process of asthma. The arrows in the figure show a pseudo-time direction of the occurrence of related biological events. The related genes involved in the GAG degradation pathway are shown in FIG. 3c.

As shown in FIG. 3d (upper panel (Cytokines) and middle panel (Immune cells)), the expression levels of asthma-related chemokines and immune cell surface antigens continued to rise with the pseudo-time series of asthma, demonstrating that the model could correctly reflect the changing sequence of various biological events after the occurrence of asthma. As shown in FIG. 3d (lower panel (GAD Degradation)), the increase of inflammatory cell infiltration leads to an increase of the expression levels of GLCE, HPSE, and lysosomal sulfatase SGSH that are responsible for the degradation of GAG In addition, the expression level of SUMF2 as an inhibitor of these enzymes was reduced. The results of biological information were verified using RNA-seq data in the mouse asthma model stimulated by OVA. As shown in FIG. 3e and FIG. 3f, as the number of OVA stimulations increases, the expression ratio of SUMF1/2 and the downstream lysosomal sulfatase SGSH thereof in lung tissue increases accordingly, but the negative control Naglu does not change substantially. The number of points in the histogram represents the number of sequenced mice. The results indicate an increase in GAG catabolism in airway epithelial cells and in the microenvironment of the airway epithelial cells affected by allergic asthma and an increase in the activity of lysosome.

### Example 2

Construction Method and Index Detection for Allergic Asthma Animal Model

SPF-grade, 4-week-old C57BL/6N female mice were treated with normal feed, free water drinking, and grew normally in a well-ventilated environment with a temperature of 20-25° C. and a humidity of 40-60%. The breeding and use of laboratory animals followed the “Reduction, Replacement and Refinement (3R)” principle and the laboratory animals were given humanitarian care.

The mice were randomly divided into an acute asthma group, an acute aggravated asthma group, a chronic asthma group, a chronic asthma plus a 2-week rest group, a chronic asthma plus a 3-week rest group, a chronic asthma plus a 4-week rest and relapse group, and an acute lung injury group, as well as control groups for each group. The mice were euthanized after the experiment. The model was judged to be successfully established according to the pathological characteristics of the lungs, such as smooth muscle hyperplasia, pulmonary fibrosis, and the number of infiltration of eosinophils, neutrophils, lymphocytes, and monocytes. The acute asthma group was marked by a large number of eosinophil infiltrations, and the acute aggravation stage was accompanied by neutrophils, monocytes and a large number of lymphocytes. The chronic asthma had a relatively small neutrophil infiltration, mainly including lymphocytes and monocytes. And there further is airway smooth muscle hyperplasia and airway remodeling. After termination of OVA stimulation, the inflammatory cell infiltration reduced gradually and finally disappeared, nevertheless the remodeled airway did not return to normal.

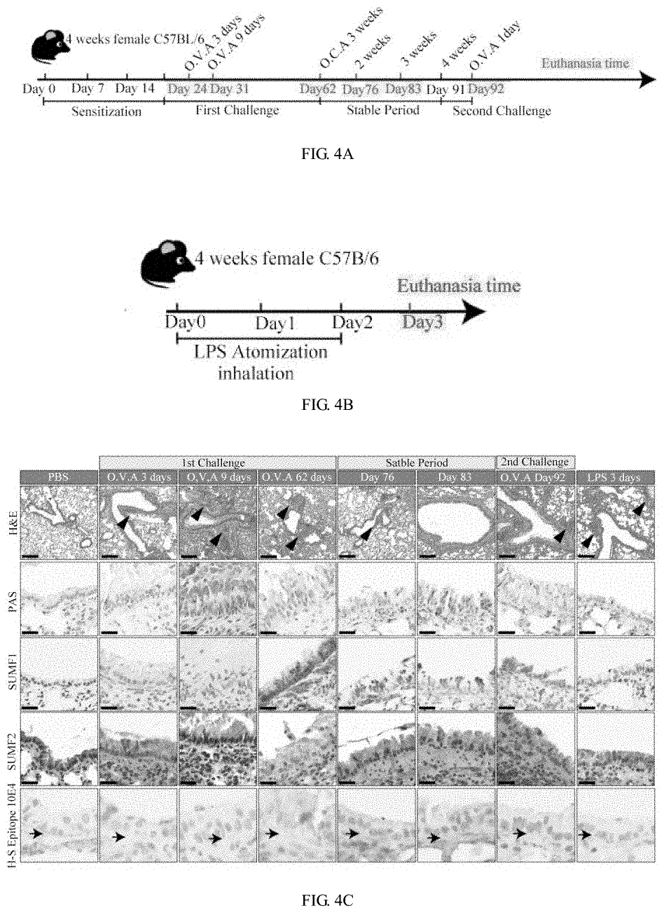

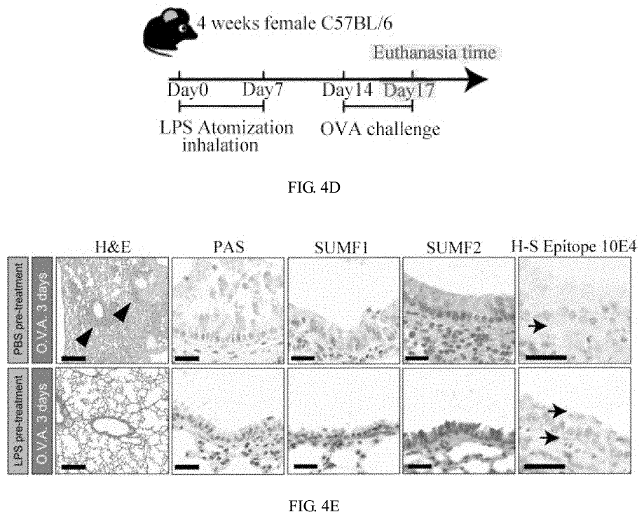

On day 0 of the experiment, mice were intraperitoneally injected with 0.2 ml of OVA+aluminum gel (40 μg of OVA, and 2 mg of aluminum potassium sulfate dissolved in 0.2 ml of PBS), and mice in control group were intraperitoneally injected with PBS of equal volume. The intraperitoneal injection was repeated on day 14 of the experiment, and the mice were subjected to nebulized inhalation of OVA to stimulate asthma using a nebulized inhalation device in combination with an anesthesia induction box on day 21 of the experiment. The acute asthma group was subjected to nebulized inhalation of 5% OVA/PBS 1 time/day for 3 days. The acute aggravated asthma group was subjected to nebulized inhalation of 5% OVA/PBS 1 time/day for 9 days. The chronic asthma group was subjected to: nebulized inhalation of 1% OVA/PBS 3 times/week for 3 weeks on the basis of acute aggravated asthma model. The chronic asthma with a 2-week or 3-week rest group was subjected to termination of nebulized inhalation for 2 or 3 weeks after successful modeling of chronic asthma. The chronic asthma with a 4-week rest and relapse group was subjected to an additional episode of nebulized inhalation of 1% OVA after resting for 4 weeks after successful modeling of chronic asthma. The acute lung injury group was subjected to one episode of nebulized inhalation of 50 μl of LPS solution (2 ng/μl) in airway after anesthetization with pentobarbital. Each control group was subjected to nebulized inhalation of PBS of equal volume. In FIG. 4a and FIG. 4b show the construction processes of mouse asthma model (acute asthma, acute exacerbated asthma, and chronic asthma) and acute lung injury model. The modeling method mentioned above was a well-established method of constructing mouse models for asthma and acute lung injury. The method was supported by plenty of literatures, and provided a modeling success rate of up to 100%.

The mouse asthma model was induced using OVA stimulation. After the modeling became successful, the mouse was euthanized at different time points. The lung tissue of the mouse was taken out, and RNA samples, protein samples and pathological samples were reserved. The inflammatory cell infiltration and SUMF1/2 expression in airway epithelial cells and the microenvironment thereof were detected using RNA sequencing in combination with an immunohistochemical staining technique, to verify the results of bioinformatics analysis. Specifically, the asthma models in each group were analyzed using a sulfation site heparan sulfate (H-S) 10E4 on the sulfatase SGSH as an indicator of the degree of sulfatase activation, and using an immunohistochemical staining technique. The results are shown in FIG. 4c. As the OVA time became longer, the expression of SUMF1 was continuously increased, while the expression of SUMF2 in mouse airway epithelial cells was continuously decreased. The ratio of the expression levels of SUMF1 to SUMF2 could not revert to normal even 3 weeks after cease of OVA stimulation.

It can be seen that as the expression ratio of SUMF1/2 the increased, a continuous decrease in the H-S 10E4 staining on the basement membrane side of epithelial cells was observed. However, the LPS stimulation had no effect on the expression of SUMF1/2 or H-S 10E4. These results indicated that repeated stimulation of allergens would lead to an irreversible increase in the expression ratio of SUMF1/2 and to desulfation of GAG on the cell membrane surface. This may result in a stronger immune response of the epithelial cells the next time when the epithelial cells were exposed to an allergen, thus forming an immune memory. In addition, reduction in SUMF1/2 expression was a unique biological event for T_{H}2 inflammation.

Since LPS pre-treatment may greatly reduce the immune response induced by OVA, the lungs of mice were lavaged with LPS, and subjected to routine acute asthma modeling. After 3 days of OVA stimulation, the mice were euthanized and the expression levels of SUMF1/2 and H-S 10E4 were determined by immunohistochemical staining technology. As shown in FIG. 4d, the mouse lungs pretreated with LPS failed to produce T_{H}2 inflammation due to OVA stimulation and the staining of SUMF1/2 and H-S 10E4 did not change. This indicated that the immediate cause for change in the expression ratio of SUMF1/2 was not the stimulation by allergen, but the repeated airway T_{H}2 inflammation.

### Example 3

Packaging, Collection, Purification, and Titer Determination of Adeno-Associated Virus

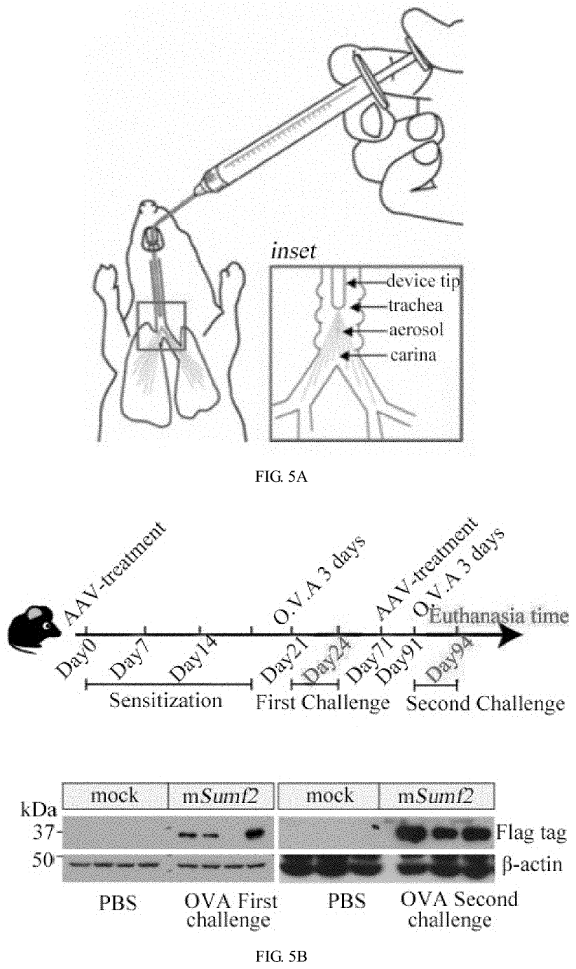

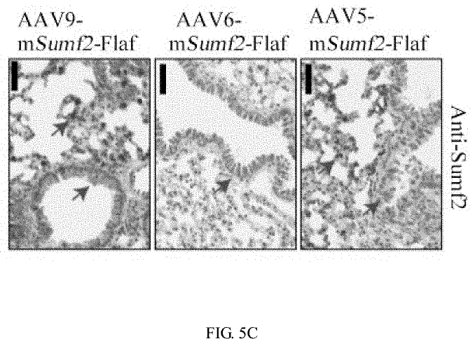

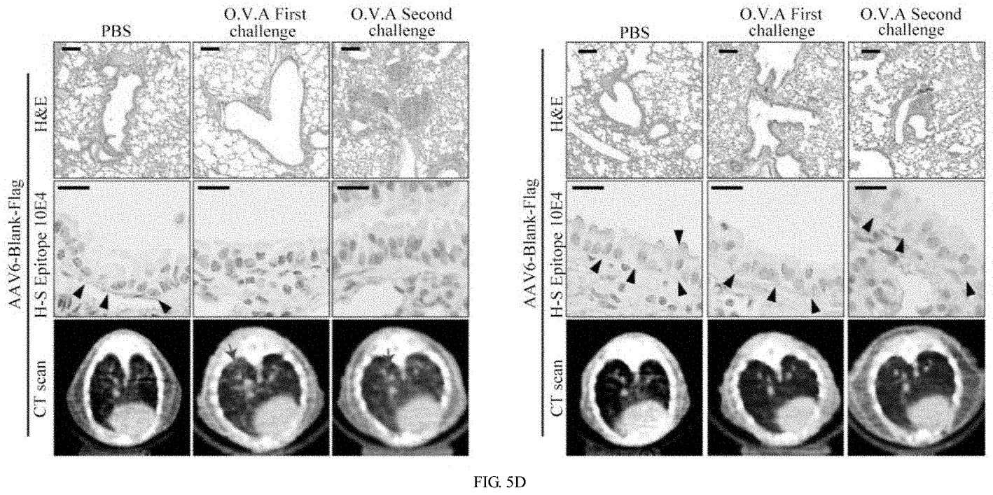

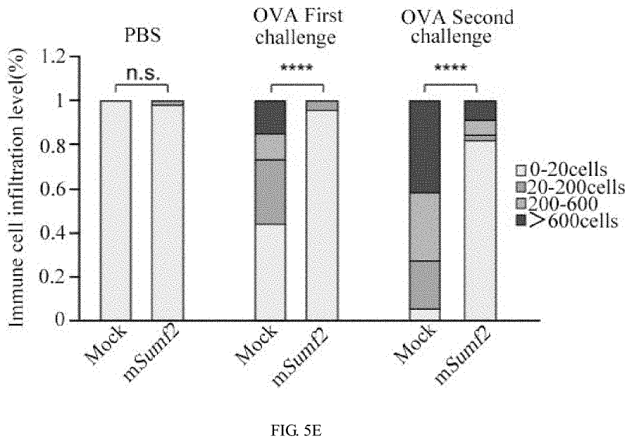

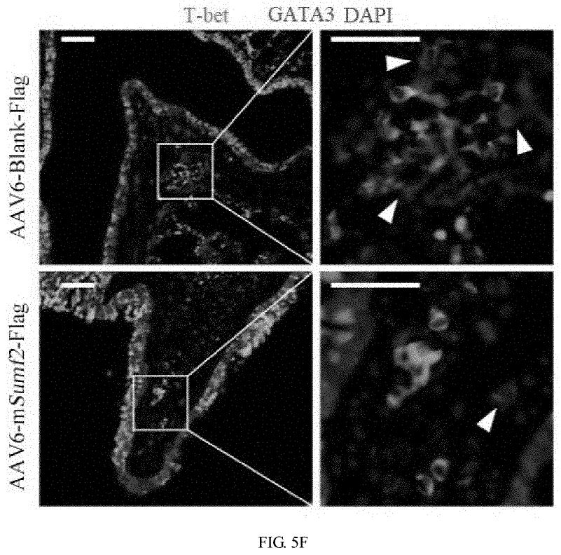

Adeno-associated virus subtypes (AAV5, AAV6, and AAV9) that were reported in the literature and were capable of binding to mouse lung cells were selected for pre-experiment. The mouse was lavaged in the airway with the AAV5-GFP, AAV6-GFP, and AAV9-GFP viruses provided by Hanbio Company, and euthanized after 21 days. And the expression of GFP in mouse lung cells was detected by immunohistochemical staining technology. As shown in FIG. 5c, AAV5 mainly bound to alveolar epithelial cells, AAV9 could simultaneously bind to both airway epithelial cells and alveolar epithelial cells, while AAV6 could well target the airway epithelial cells. Therefore, AAV6 was selected as a vector for the delivery of SUMF2 gene.

In this example, AAV6 subtype that can well target mouse lung airway epithelial cells to overexpress SUMF2 was selected. The AAV6 genome included two inverted terminal repeats (ITR) and two open reading frames (ORF), namely Cap and Rep, and Rep could be translated into a variety of proteins necessary to the life cycle of AAV. In the AAV packaging system, Rep and Cap were replaced with the target gene SUMF2-3flag/3flag (the mouse SUMF2 cDNA had a sequence shown in SEQ ID NO: 2), and co-transfected with the plasmid expressing the Rep and Cap genes to produce the AAV virus containing target genes.

The AAV packaging system included 3 types of plasmids, which were a shuttle plasmid AAV6-SUMF2-3flag/3flag for inserting exogenous genes, a pAAV6-RC plasmid for encoding Rep and Cap proteins, and a pHelper plasmid for expressing adenovirus proteins on which AAV depended (the map of the AAV packaging system was shown in FIG. 8). The above 3 types of plasmids were co-transfected into a virus packaging cell AAV-293 (with a confluence of 50-70%, and free of mycoplasma contamination) by conventional methods, and transfected in a T75 culture flask containing the reagents as follows: pAAV6-RC 10 μg, pHelper 20 μg, AAV6-SUMF2-3flag or AAV6-3flag (control plasmid) 10 μg, and LipoFiter 80 The virus packaging cell AAV-293 was cultured to give an AAV virus particle carrying a foreign inserted gene.

Six hours after the transfection of AAV-293 cell, the system was transferred to a DMEM medium containing 10% fetal bovine serum. All the cells were collected after 72 hours, frozen and thawed three times using liquid nitrogen. The supernatant was to be collected and purified using a Biomiga purification column. The AAV virus sample to be tested was digested routinely with DNase I and proteinase K, titer of the virus was determined by real-time fluorescent quantitative PCR (using a plasmid with a known copy number as a standard), dispensed and stored at −80° C. The specific method was as follows:

A. Determination of titer of a virus

1. Preparation of AAV6 sample

AAV6 virus sample was treated using DNase I and proteinase K to prepare a PCR template.

(1) DNase I reaction system

DNase I, 10 μL

DNase I Buffer, 6 μL

AAV sample, 3 μL

dd H₂O, 41 μL

(2) the sample was treated in a 37° C. water bath for 40 min, and inactivated in a 100° C. metal bath for 5 min; and

(3) three μL of proteinase K was added, the sample was allowed to be treated in a water bath at 55° C. for 40 min and inactivated in a metal bath at 100° C. for 5 min for further use.

2. Preparation of a standard

A plasmid having a known copy number was used as a standard and was gradiently diluted to final concentrations of 10⁸, 10⁷, 10⁶, and 10⁵ copies/ml.

3. PCR reaction system

2×SYBR Green mix 10 μl,

Primer (F or R) 1 μL

Standard plasmid/virus template 1 μL

ddH₂O 8 μL

Total 20 μl,

| | Forward primer: |
|---|---|
| (SEQ ID NO: 3) | |
| | acgctatgtggatacgctgc; |
| | Reverse primer: |
| (SEQ ID NO: 4) | |
| | cgggccacaactcctcataa. |

4. qPCR reaction program

UDG activation, 50° C. for 2 min

Polymerase activation, 95° C. for 2 min

Denaturation, 95° C. for 15

Annealing/extension, 60° C. for 1 min (40 cycles);

65-95° C. melt curve.

Results

The results for virus determination of standards of different concentrations are shown in Table 1.

TABLE 1
Virus gradient results at different concentrations

| Concentration | 10⁸ | 10⁷ | 10⁶ | 10⁵ |
|---|---|---|---|---|
| Mean | 9.72 | 13.03 | 15.34 | 19.10 |
| CT1 | 9.78 | 12.94 | 15.65 | 19.31 |
| CT2 | 9.53 | 13.01 | 15.15 | 19.09 |
| CT3 | 9.86 | 13.14 | 15.22 | 18.92 |

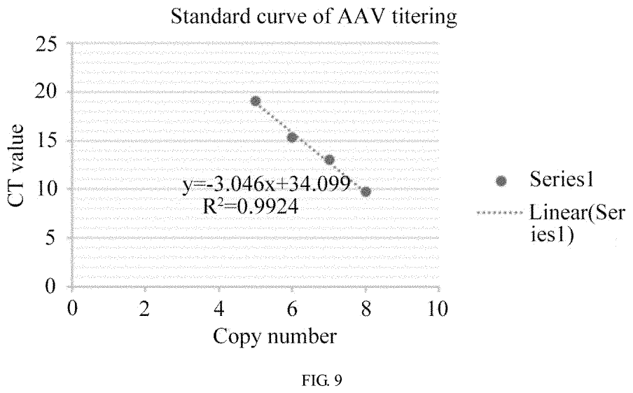

The standard curve is shown in FIG. 9. The function formula and R-square value of the standard curve were obtained by taking the mean CT value in each group of standards as ordinate Y, and a corresponding logarithm of a copy number of the CT value as an abscissa X.

B. Test results for AAV virus samples

TABLE 2
Test results for control virus and virus containing SUMF gene

| | AAV6-3flag | AAV6-SUMF2-3flag |
|---|---|---|
| Mean | 8.25 | 8.76 |
| CT1 | 8.24 | 8.81 |
| CT2 | 8.15 | 8.75 |
| CT3 | 8.37 | 8.73 |

The mean Ct values of the AAV samples were put into the formula, a copy number X of the added AAV template was calculated and converted into the titer. A conversion formula is shown in the following Formula I:

AAV virus titer=10^{x}×40000 (dilution multiple) vg/mL Formula I

Upon calculation, the titer of AAV6-SUMF2-3flag was about 1×10^{12.8} vg/mL, and the titer of AAV6-3flag was about 1×10^{12.9} vg/mL.

### Example 4

Construction of Mouse Model of Gene Therapy Targeting SUMF2

Mice were infected with AAV6-SUMF2 at different time points to simulate the drug use after first exposure to allergens and airway hyperresponsiveness.

1. Thirty two (32) 4-week-old female C57BL/6 mice were randomly divided into SUMF2 overexpression group and a control group. Mice were subjected to OVA sensitization on day 0 and day 14 of the experiment. A purified adeno-associated virus and a control virus (packaged by AAV6-3flag plasmid) were injected into the airways of SUMF2 overexpression group and the control group (containing approximately 0.5×10¹¹ viral particle genome copies) through an airway nebulizer on day 1. The mice were subjected to nebulized inhalation of a PBS liquid with 5% OVA for three days on day 21, subjected to pulmonary CT and sacrificed with CO₂ on day 24, and lung tissues were harvested for pathological analysis.

2. An acute exacerbated allergic asthma model was constructed using 32 4-week-old female C57BL/6 mice (using the method same as in Example 2), and nebulized inhalation was stopped for a period of time to allow the mice enter a stable period of asthma. The adeno-associated virus and the control virus were injected into the airways of the experiment group and the control group. The mice received nebulized inhalation of a PBS liquid with 5% OVA for three days from day 21, and the mice were subjected to pulmonary CT and sacrificed with CO₂ on day 24; and lung tissues were taken out for pathological analysis.

Results

The adeno-associated virus (AAV) having specific affinity to epithelial cells was used as a gene therapy vector to infect the respiratory epithelial cells of mice through nebulized inhalation, thereby forcibly increasing the expression level of SUMF2 (FIG. 5a). Mice were infected at two different timepoints, i.e., before the first OVA stimulation and the second OVA stimulation to simulate the state of the first exposure to allergens and airway hyperresponsiveness (upper part of FIG. 5b). The overexpression of SUMF2 was successfully detected by Western Blot (lower part of FIG. 5b). Through the experiment it was confirmed that the AAV6 vector that infected the airway epithelial cells could target epithelial cells (FIG. 5c) only. As shown in FIG. 5d, AAV6-SUMF2 therapy significantly reduced immune cell infiltration (FIG. 5e) and reversed the sulfidation state of GAG on the cell surface, compared with the control group. CT scanning showed that the appearance of interstitial pneumonia such as ground-glass-like and grid-like shadows in both lungs was dramatically reduced. Whether it was the firstly sensitized by OVA had little effect on the treatment results. Since asthma was not induced until 20 days following the administration of AAV6-SUMF2, the AAV6-SUMF2 could be used as a treatment method to prevent asthma and used for disease management in the stable period of asthma. The results of immunofluorescence staining of lung tissue showed that GATA3 cells were significantly reduced and the T_{H}2 allergic reaction was suppressed (FIG. 5f).

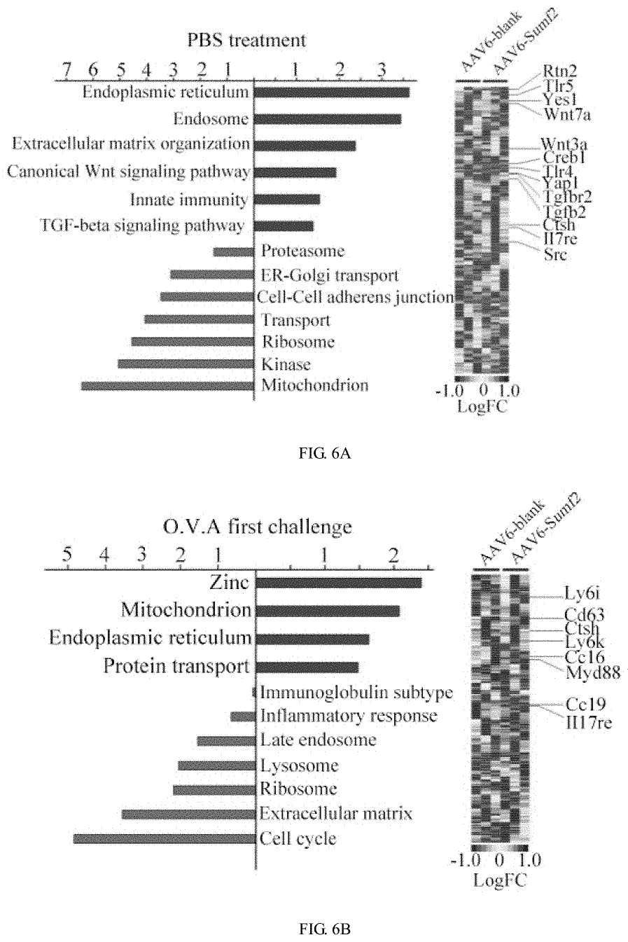

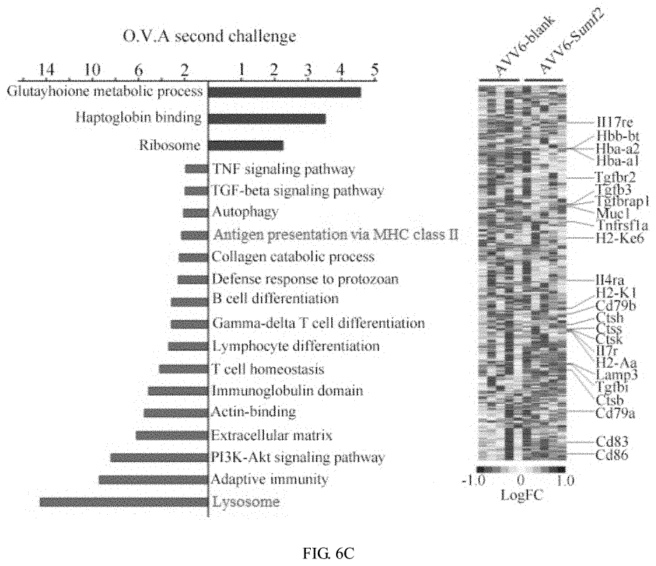

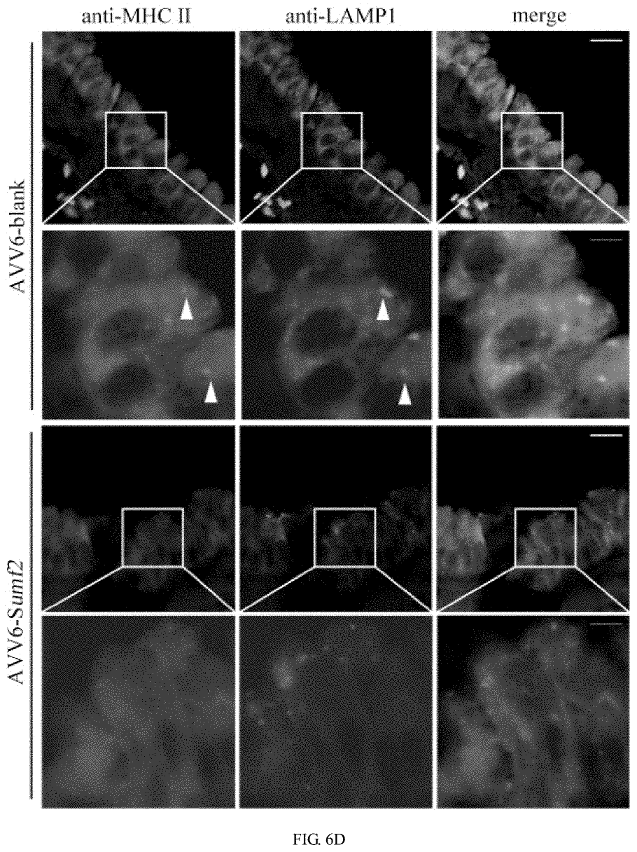

In order to explore the specific mechanism of SUMF1/2 regulating the T_{H}2 response, RNA-seq was conducted on the lung tissues of AAV6-mSUMF2 gene-treated mice and control mice at different time points, and gene expression enrichment analysis was performed using differential genes between groups. The results are shown in FIG. 6a. SUMF2 induced overexpression of a variety of cytokines in the lung tissues of mice without OVA stimulation and activated the Wnt signal. This might be due to the increased level of GAG sulfation on the cell surface, leading to excessive accumulation of secreted proteins on the cell membrane surface and to activation of related receptors. In the OVA stimulation group, especially during the second OVA stimulation, SUMF2 therapy blocked almost all events of the T_{H}2 response (FIG. 6b and FIG. 6c). It should be noted that AAV6-mSUMF2 inhibited the expression of lysosome and MHC II-related genes. Further studies had found that OVA could induce epithelial cells to express MHC II and increase co-localization thereof with the lysosomal protein LAMP1 (FIG. 6d), dotted staining shown by white triangles). However, the therapy using AAV6-mSUMF2 greatly reduced the punctate staining of MHC II in epithelial cells, and lysosomal storage appeared in the cells. According to the existing knowledge, the co-localization of MHC II and LAMP1 was usually seen in the process of antigen processing and presentation. The lysosomal storage might be resulted from the inhibition of sulfatase in the lysosome by AAV6-mSUMF2.

Described above are merely preferred implementations of the present disclosure. It should be noted that a person of ordinary skill in the art may further make several improvements and modifications without departing from the principle of the present disclosure, and such improvements and modifications should be deemed as falling within the protection scope of the present disclosure.

What is claimed is:

1. A method for preventing and/or treating an allergic asthma attack and reducing airway hyperresponsiveness, wherein the method comprises a step of targeting SUMF2 gene of a patient.

2. The method according to claim 1, wherein SUMF2 gene has the nucleotide sequence shown in SEQ ID NO: 1.

3. The method according to claim 1, wherein SUMF2 gene is used jointly with a gene vector for gene therapy for preventing and/or treating an allergic asthma attack and reducing airway hyperresponsiveness.

4. The method according to claim 3, wherein the gene vector comprises an adeno-associated virus packaging system.

5. The method according to claim 4, wherein the adeno-associated virus packaging system comprises a shuttle plasmid for inserting exogenous genes, a pAAV6-RC plasmid for encoding Rep and Cap proteins, and a pHelper plasmid for expressing adenovirus proteins.

6. A drug for preventing and/or treating an allergic asthma attack and reducing airway hyperresponsiveness, wherein the drug comprises a component targeting SUMF2 gene in airway epithelial cells.

7. The drug according to claim 6, further comprising a gene vector for SUMF2 gene.

8. The drug according to claim 7, wherein the gene vector comprises an adeno-associated virus packaging system.

9. The drug according to claim 8, wherein the adeno-associated virus packaging system comprises a shuttle plasmid for inserting exogenous genes, a pAAV6-RC plasmid for encoding Rep and Cap proteins, and a pHelper plasmid for expressing adenovirus proteins.

10. The method according to claim 3, wherein SUMF2 gene has the nucleotide sequence shown in SEQ ID NO: 1.
